(19) United States
(12) Patent Application Publication
(10) Pub. No.: US 2002/0128708 A1
(43) Pub. Date: Sep. 12, 2002

(54) Annuloplasty system

(76) Inventors: William F. Northrup III, Edina, MN (US); Joanne B. Northrup, Edina, MN (US)

Correspondence Address: William M. Hienz III, Dicke, Billig & Czaja, P.A., 701 Building, Suite 1250, 701 Fourth Avenue South, Minneapolis, MN, 55415, US

(21) Appl. No.: 09/733,224

(22) Filed: Dec. 8, 2000

Related U.S. Application Data
(60) Provisional application No. 60/170,085, filed on Dec. 9, 1999.

(51) Int. Cl. A61F 2/24

(52) U.S. Cl. 623/2.37; 606/232

(57) ABSTRACT

An apparatus for stabilizing an anatomical structure includes a combination of repeating rigid elements within a single flexible element. While applicable to routine open surgical techniques and to a variety of anatomical structures, the system is especially applicable to minimally invasive endoscopic/robotic mitral valve repair. An annuloplasty system according to the invention is immediately intuitive, logical and simple to use.

### CROSS-REFERENCE TO RELATED APPLICATIONS

The subject matter of this application is related to the subject matter of U.S. provisional patent application Ser. No. 60/170,085, filed Dec. 9, 1999, priority to which is claimed under 35 U.S.C. §119 and which is incorporated herein by reference.

The subject matter of this application is also related to the subject matter of commonly assigned U.S. Pat. Nos. 5,593,424, 5,709,695, and 5,961,539, all of which are incorporated herein by reference.

### BACKGROUND OF THE INVENTION

b 1. Field of the Invention

Aspects of the invention relate to devices and methods for repairing anatomical structures, such as heart valves. In particular, devices and methods according to embodiments of the invention allow for substantially permanent, substantially rigid fixation of either part or all of the circumference of a heart-valve annulus, for example, the mitral annulus. Embodiments of the invention are equally desirable in minimally invasive and in conventional surgical situations, and are especially, though not exclusively, useful in situations involving robotic endoscopy.

2. Description of Related Art

Some key opinion leaders in the field of heart-valve repair believe a need exists for a rigid element that is permanently fixed to either a portion of or the entire circumference of a heart-valve annulus. In particular, Carpentier, who arguably has been and likely still is the primary opinion leader worldwide, began his extensive career repairing mitral valves with an entirely rigid, completely circumferential ring. Carpentier rings are designed to be permanently fixed to the mitral annulus after stabilizing and/or reducing its circumference. See, for example, Carpentier, A., “La valvuloplastie reconstructive une nouvelle technique de valvuloplastic mitrale,” Presse Med, 1969; 77: 251-3, which is incorporated herein by reference.

Like other opinion leaders, Carpentier has believed that a rigid element is necessary along the anterior annulus of the mitral valve in order to accomplish true “remodeling” of the mitral annulus. These leaders have preferred fixation of the entire mitral annulus to a ring that always is rigid along the anterior annulus, and in fact these leaders continue to implant completely rigid rings. (e.g. Carpentier Classic, Baxter). Recently, however, Carpentier has developed an annuloplasty ring that is rigid along the anterior mitral annulus but is relatively flexible along the posterior mitral annulus. (e.g. Carpentier Physio, Baxter). See, for example, Carpentier, A. et al., “The ‘Physio-Ring’: An Advanced Concept in Mitral Valve Annuloplasty,” Ann. Thorac. Surg. 1995: 60: 1177-86, which is incorporated herein by reference.

Other key opinion leaders favor annuloplasty rings that are completely flexible. The Duran ring (Medtronic) is a completely flexible annuloplasty ring that is fixed to the entire circumference of the mitral annulus. This complete-ring device became available in the 1970's. More recently, Cosgrove (Baxter) has developed a completely flexible annuloplasty ring that is fixed only to the posterior mitral annulus, forming a partial ring. Debate continues as to whether true heart-valve “remodeling” actually requires any rigid element in the annuloplasty ring to which the mitral annulus is permanently fixed.

The three U.S. patents incorporated by reference above provide a more recent approach, as will now be described. A basic aspect of this approach involves a plurality of suture support segments or platforms, like that shown generally at 10 in FIGS. 1A-1B. Each individual suture support segment 10 includes upper surface 12, lower surface 14, and opposite sides 16, 18. Two suture holes or apertures 24, 26 extend through suture support segment 10. Each suture aperture 24, 26 preferably includes chamfered portions 28 at upper surface 12 and lower surface 14 of segment 10, to reduce abrasion and consequent fraying or other damage to sutures passing through apertures 24, 26. Apertures 24, 26 also preferably include substantially straight sidewalls, as shown, and preferably are large enough to accommodate a 2-0 suture and swedged-on needle. Although suture support segment 10 may be of any suitable overall shape, the rounded-end rectangle shape shown in FIGS. 1A-1B is particularly desirable.

Suture apertures 24, 26 are separated by a center-to-center distance D, which preferably is about 5.0±3.0 mm. Segment 10 preferably has a width W of about 3.0 mm±1.0 mm, a thickness T of about 1.0 mm, and a length L of about 7.0 mm. Of course, other dimensions to fit a particular surgical application are also contemplated.

Suture support segment 10 can be made of any suitable material that is preferably inert, non-corrosive, non-thrombogenic and biocompatible with blood and tissue. A material already approved by the FDA for intra-vascular use is preferred, such as titanium, or an alloy of titanium such as a medical-grade titanium-aluminum-vanadium alloy.

Suture support segment 10 should be non-deformable in its long axis, and therefore substantially rigid. Each suture support segment 10 preferably accommodates a single horizontal mattress suture incorporating a portion of the circumference of a tissue annulus beneath it.

As described in the above-identified patents, multiple segments 10 are appropriately spaced and positioned with respect to a tissue annulus, e.g. during an annuloplasty procedure, and then covered with a flexible material, such as autologous pericardium, to create an annuloplasty ring. The dilated tissue annulus of e.g. a mitral or tricuspid heart valve can be precisely plicated and/or stabilized to an exact dimension of several, individual segments 10, with consistent intervals defined between segments 10 and a specific length of flexible ring material. The annuloplasty ring can be either partial or complete, and has overall flexibility.

FIG. 2 shows how multiple segments 10 are used to restore tissue annulus 3 0 to its original, healthy shape 32. Mattress sutures 34 are properly placed in tissue annulus 30, as shown, passing through pericardial strip 36 and respective segments 10. Segments 10 will cause exact, consistent spacing S between mattress sutures 34 along the length of pericardial strip 36. Plication of tissue annulus 30 within the two limbs of each respective mattress suture 34 is represented at 38, for example.

As shown in FIGS. 3-4, pericardial strip 36 is pushed down to tissue annulus 30, shiny side down, so that pericardial strip 36 lies directly on tissue annulus 30. Each segment 10 is also pushed down to pericardial strip 36 so that each segment 10 lies on top of it. Each mattress suture 34 is then tied firmly over each segment 10, and the knot tails cut. Extra pericardial strip 36 is then folded tightly back from mitral orifice 40, according to one embodiment, over the top of segments 10 and their knots, as shown beginning at 42. The shiny side of the pericardium will then be on top of the smooth folded edge of pericardial strip 36 immediately adjacent to mitral orifice 40. The free edges of the two layers of the pericardial strip 36 will be immediately adjacent to the supra-annular atrial endocardium along the back edges of segments 10. Extra pericardium is then trimmed away at the ends of the two trigonal segments and along the back edge of all segments 10, leaving just enough for a running suture line 44, shown in FIG. 4. The folding process creates a two-layer pericardial tube 46.

Other embodiments of the inventions described in the patents provide a delivery system for delivering the segments to the anatomical structure in a precise, evenly spaced manner.

The above-described embodiments are believed to provide significant advantages over commonly used annuloplasty rings and the like. Still, the surgeon must “build” the ring from the bare segments. Further, for use in minimally invasive procedures, individual segments would have to be passed through an endoscope or other tool. A desire has arisen to provide additional advantages in view of these issues.

### SUMMARY OF THE INVENTION

In view of the above considerations, a new system for stabilizing an anatomical structure is described, e.g. an annuloplasty system, comprising a combination of repeating rigid elements within a single flexible element. While applicable to routine open surgical techniques and to a variety of anatomical structures, it is especially applicable to minimally invasive endoscopic/robotic mitral valve repair.

Material and Methods: One particular annuloplasty ring according to the invention includes thirteen separate, rigid, titanium suture platforms, arranged end-to-end and enclosed in a single flexible polyester tube. The suture platforms are of an oval shape and are about 7× about 3× about 0.5 mm, each with two suture holes about 3 mm apart. The two ends of a single mattress suture from the tissue annulus are passed through the suture holes in each suture-platform, allowing for plication of the underlying tissue while preventing plication of the surrounding flexible ring material. The broad surface area of the suture platforms, with the suture platform inside the suture loop, also provides a buttress function to ensure firm apposition of the ring to the tissue annulus with predictable knot security.

The suture platforms are separated by about 3 mm, according to one embodiment, with transverse polyester stitching in the overlying polyester tube, creating thirteen compartments or pockets exactly 10 mm in length, according to one example. Additionally, the suture platforms are stitched to the overlying polyester tube through the suture holes in the long axis of the ring, providing a firm union and an external guide to the placement of each end of the individual mattress suture. The ring is affixed to an easily detachable semi-flexible sizer/delivery system for sizing the perimeter of the free margin of the unfurled anterior mitral leaflet, for example as disclosed in the above-mentioned U.S. Pat. No. 5,961,539. Since the transverse stitching in the ring occurs e.g. every 10 mm, it functions as a ruler, providing the surgeon with a perimeter dimension in cm of the free margin of the anterior mitral leaflet. The inter-trigonal or inter-commissural dimensions can also be included if the measurement of the complete circumference of the leaflet is desired.

Results: An annuloplasty system according to aspects of the invention is immediately intuitive, logical and simple to use, employing one suture in the tissue annulus per rigid suture-platform per cm of ring. The mattress sutures are first placed to correspond to the first and last suture-platforms and then equally spaced around the annulus, according to one embodiment. If, for any reason, additional sutures are required, they can be brought up through the transverse stitching at right angles to the long axis of the ring without any resultant foreshortening of the ring.

Sizing the circumference of the anterior mitral leaflet eliminates obligate assumptions of leaflet height and area based on an inter-trigonal or inter-commissural dimension, as is believed the case with all commercial annuloplasty sizers. As such, it eliminates “making do” with a “best fit” between width and height of a small number of predetermined generic sizers. An infinite number of sizes are possible with this annuloplasty system by simply adjusting the precise position of the first and last suture-platforms. Accordingly, it will always be possible to make the ring fit the valve, eliminating the need to ever make the valve fit the ring.

The ring can be cut to any specified length, e.g. in whole centimeters, and, therefore, is customizable as either a partial or complete ring. Since the ring size is determined by the actual circumference dimension of the anterior mitral leaflet (and not some arbitrary relationship of width and height), it is also customizable for any size or shape mitral valve. Since the precise dimension of the anterior mitral leaflet can be determined in every case, and since the ring cannot deform in the long axis, a precise plication of the tissue annulus is possible in every case.

Conclusions: This unique annuloplasty system of one size, with a sizer/delivery system initially attached to the ring, can be straightened for a single-pass introduction through a small endoscope. The sizer/delivery system and ring can then be reconfigured in the thorax to match the circumference of the unfurled anterior mitral leaflet and, thus, to determine the appropriate size ring, without the need to modify the ring or the sizer or make a counter-incision. Accordingly, it has unique applicability to minimally invasive endoscopic/robotic techniques.

Other features and advantages according to the invention will be apparent from the remainder of this disclosure.

### BRIEF DESCRIPTION OF THE DRAWINGS

Preferred embodiments of the invention will be described with respect to the figures, in which like reference numerals denote like elements and in which:

FIG. 1A is a top view of a suture support segment according to an embodiment of the invention.

FIG. 1B is a cross-sectional view along line 1B-1B of FIG. 1A.

FIG. 2 is a view showing placement of mattress sutures through a pericardial strip and through suture support segments, according to an embodiment of the invention.

FIG. 3 is a view showing segments tied over pericardium, and pericardium in the process of being folded over the segments to form a tube, completely enclosing the segments over 360 degrees, according to an embodiment of the invention.

FIG. 4 is a view showing a completed posterior annuloplasty ring constructed of suture support segments and autologous pericardium, the pericardium completely enclosing the segments over 360 degrees to form a tube, according to an embodiment of the invention.

FIG. 5 is an overhead view of an annuloplasty system according to an embodiment of the invention.

FIG. 6 is a partial side view of the FIG. 5 annuloplasty system.

FIG. 7 is an end view of the FIG. 5 annuloplasty system.

FIG. 8 is a side view of an endoscope usable according to embodiments of the invention.

### DETAILED DESCRIPTION OF PREFERRED EMBODIMENTS

According to embodiments of the invention, a preferred number of suture platforms, e.g., thirteen titanium suture platforms, each include two suture holes of about 1 mm diameter spaced about 3 mm apart. Of course, other dimensions and materials are contemplated according to embodiments of the invention. According to one embodiment, all of the suture platforms are of a single size and are generally identical.

A fabric tube, e.g. a white polyester tube, completely encloses the suture platforms. According to one embodiment, tubes of a single size can be used to fit a variety of patients, and each tube is formed of a single piece of polyester or other fabric. According to one embodiment, the fabric is generally porous and substantially prevents direct contact between the suture platforms and blood or other outside biological materials. Other embodiments of suture-platform covering materials are described in the above-noted U.S. patents.

A fixation/guide suture, preferably black in color or otherwise distinct from the tube, fixes each suture platform to the polyester tube, eliminating the need for an additional bonding agent to hold the suture platform in place. Thus, only two materials are present in the system, according to the illustrated embodiment: the titanium or other metal/material of the suture platform, and the polyester or other fabric/material of the surrounding tube. The fixation/guide suture also directs the surgeon's needle to the two holes of each covered suture platform, for placement of the annuloplasty suture. A bonding agent can additionally or alternatively be used to secure the suture platform with respect to the tube. An additional or alternative internal or external guide feature also can be used, to accurately guide and direct suture placement.

An inter-segmental suture, e.g. of black polyester, is disposed between and/or forms individual links of the tube, between the suture platforms. The inter-segmental sutures preferably define a fixed, repeating interval of e.g. 10 mm, according to one embodiment, allowing accurate and precise delivery of the spaced suture platforms. Other sizing/delivering advantages and features used in conjunction with the invention are described in e.g. the above-noted U.S. Pat. No. 5,961,539.

The inter-segmental sutures cause each suture platform to be enclosed, or partially enclosed, within the polyester tube. More specifically, the inter-segmental sutures cause each suture platform to be enclosed within its own link or pocket or segment of the tube, according to the illustrated embodiment. Further, they allow the surgeon to cut the ring at any desired length, e.g. just outside a desired intersegmental suture, to size the overall tube/ring to a particular valve circumference or portion thereof. The suture platform of the new end remains enclosed within its link of the tube.

The tube link containing the middle suture platform preferably includes an additional or alternative external marker, e.g. a white fixation/guide suture instead of a black one, for orientation and accurate delivery of the tube. The tube preferably is of a low profile, according to embodiments of the invention, and provides a scaffold for a biomaterial (e.g. untreated autologous pericardium) with the absence of a ring template or holder.

Significantly, embodiments of the invention can be completely fabricated at the manufacturing facility prior to marketing. The surgeon does not complete or “build” the ring from the bare segments; instead the segments are spaced and delivered as a unit in the tube. The tube then can be cut to a desired length, as described above.

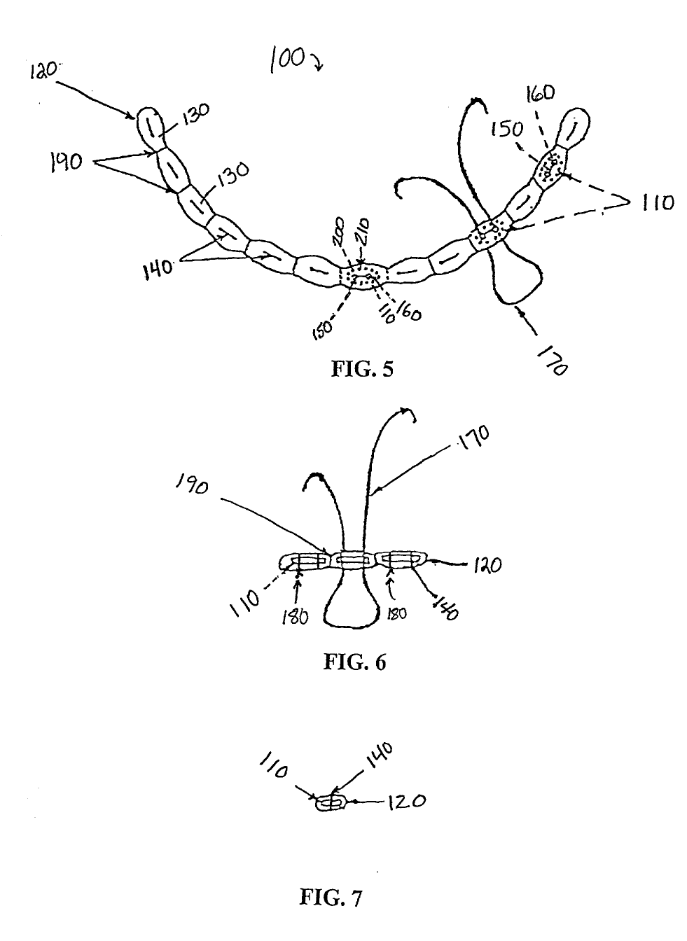

FIG. 5 shows one particular embodiment of an annuloplasty system according to the invention. The system includes apparatus 100 for stabilizing an anatomical structure, such as a tissue annulus, for example. Apparatus 100 includes a plurality of discrete suture support platforms 110, constructed and arranged for disposition at the anatomical structure to stabilize the anatomical structure, generally as described above. Covering material 120, such as a white polyester tube, is constructed and arranged to cover the plurality of discrete suture support platforms 110. Covering material 120 is formed into a plurality of pockets 130, which in the illustrated embodiment number thirteen and are generally identical. Of course, as described elsewhere herein, specific types of covering material other than polyester and in other colors are also contemplated. Additionally, although thirteen pockets 130 are illustrated, any number of pockets may be provided to suit a particular environment or situation. At least one discrete suture support platform 110 is contained within each pocket 130, with exactly one such platform 110 being contained in each pocket 130 according to the illustrated embodiment.

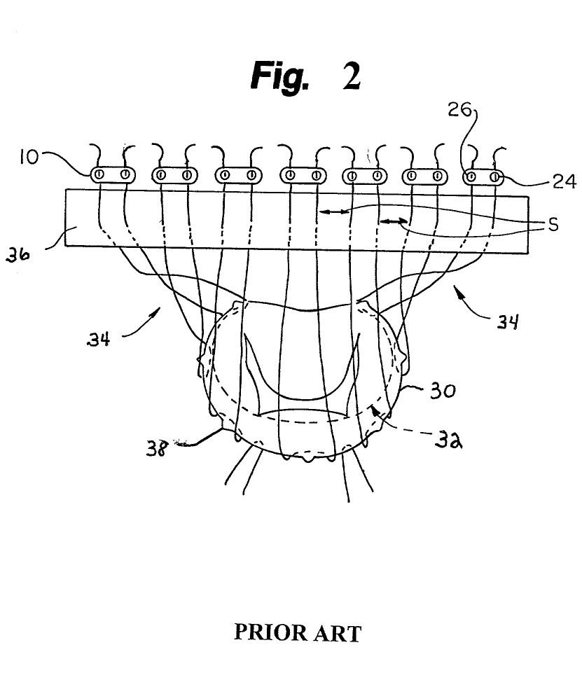

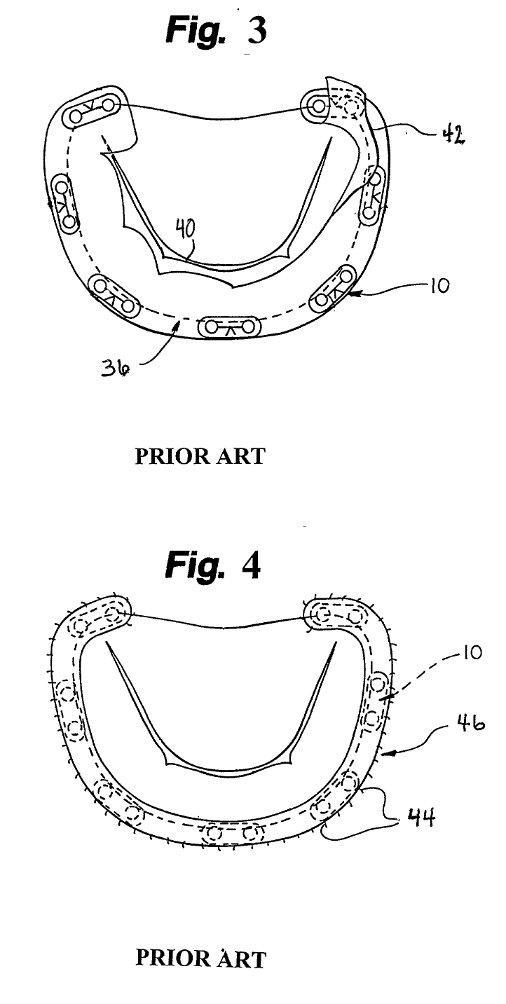

Each suture support platform 110 is constructed and arranged to support and receive suture, extending from platform 110 through covering material 120. This suture serves to secure platform 110 with respect to the anatomical structure at a precise location, generally in the manner described with respect to FIG. 2, for example.

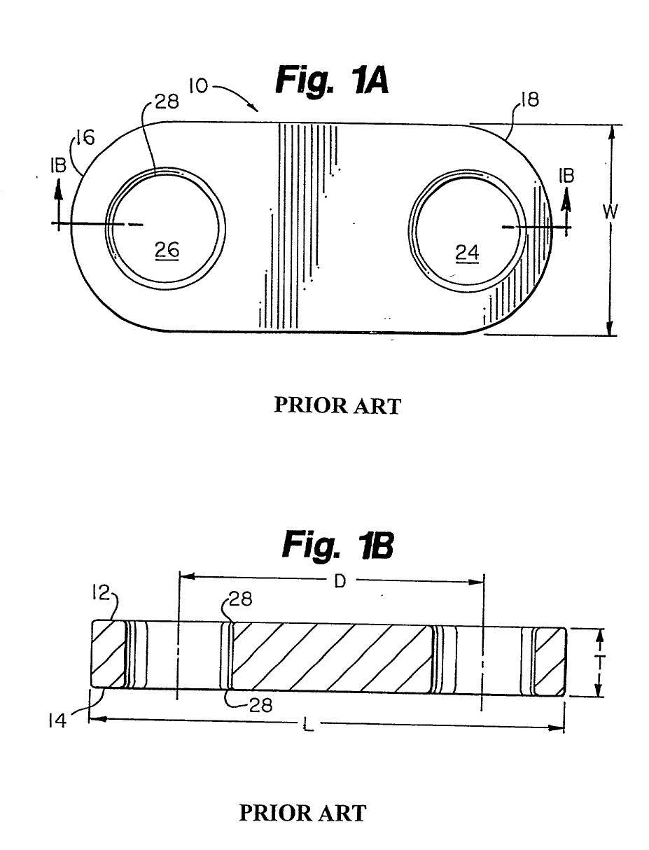

Each suture platform is attached to covering material 120 in any manner that generally prevents their relative movement. According to one embodiment, this attachment is accomplished by fixation suture 140. Suture 140 passes through e.g. two suture apertures 150, 160 extending through platform 110, in a manner akin to that described with respect to apertures 24, 26 in FIGS. 1A-1B. As generally described in the above-referenced U.S. patents, apertures 150, 160 of platforms 110 are constructed and arranged to create a plication region in the anatomical structure beneath each platform, so as to reduce the length of the anatomical structure by a precise amount along the line.

Although using fixation suture 140 presents several advantages, to be described, it is also contemplated to use e.g. a bonding agent between covering material 120 and platform 110 to secure the one with respect to the other. According to one embodiment, fixation suture 140 is black in color, to present a clearly visible contrast with respect to the underlying white covering material 120. Of course, other color schemes for covering material 120 and fixation suture 140 are contemplated, preferably providing a high degree of contrast.

The contrast between suture 140 and underlying covering material 120 provides a clear visual indication of the location of apertures 150, 160 in suture platform 110. This visual indication readily allows the surgeon to accurately place mattress suture 170 through apertures 150, 160, even though covering material 120 is generally opaque. Thus, fixation suture 140 also acts as a guide suture, to guide the placement of mattress suture 170 through covering material 120 and apertures 150, 160. To further provide a visual indication, a knot 180 (FIG. 6) with which fixation suture 140 is tied can be disposed directly above/adjacent one of the apertures 150, 160, thereby indicating their location. In the absence of a visible knot or specific knot placement, the visible ends of fixation suture 140 serve to indicate where the underlying apertures 150, 160 are located.

Other mechanisms for indicating the location of apertures 150, 160 are contemplated. For example, covering material 120 can include multiple lines, arrows, dots or other printed visual indicators. Additionally, covering material 120 can be transparent or otherwise allow apertures 150, 160 to indicate their location directly. In other words, apertures 150, 160 can be their own visual indicators of their location. In any of these examples, including the example of a fixation suture, it can be said that covering material 120 supports a visual indication of the location of apertures 150, 160.

According to an embodiment of the invention, covering material 120 also supports intersegmental sutures 190. Intersegmental sutures 190 are constructed and arranged between platforms 110 to form pockets 130. According to one embodiment, intersegmental sutures 190 are of a contrasting color relative to covering material 120. Intersegmental sutures comprise transverse polyester stitching, according to one embodiment, in the overlying polyester tube 120, creating the thirteen pockets or compartments 130 illustrated in e.g. FIG. 5. According to one embodiment, the distance between sutures 190 is exactly 10 mm, so that each compartment 130 is exactly 10 mm in length. Since each transverse intersegmental stitching/suture 190 then occurs with respect to covering material 120 every 10 mm, it functions as a ruler, providing the surgeon with a perimeter dimension in centimeters, according to one embodiment, of e.g. the free margin of the anterior mitral leaflet. Thus, intersegmental sutures 190 serve as sizing features, which can indicate the length of the anatomical structure to which apparatus 100 is being applied.

Of course, other color contrast schemes and visual indicators are contemplated for use instead of or in addition to intersegmental suture 190. According to one example, covering material 120 can include intersegmental transverse marks, such as lines, dots, arrows, printing, indentations or other texturing, or the like. One advantage of using suture 190, on the other hand, is that suture 190 tends to draw the edges of pockets 130 into a narrowed portion or waist portion which (1) partially closes or completely closes the ends of the respective pockets 130, (2) provides additional visual indication of where the pockets begin and end, (3) provide greater structural soundness to the overall tube formed by covering material 120, and, relatedly, (4) provides an anchor point for additional suture, if needed to better secure the completed tube to the anatomical structure in question. More specifically, at the waist between suture platforms 110, an additional mattress suture in the tissue annulus or other anatomical structure can be placed, with both ends of the additional suture brought up through the tube 120 radially, that is, at right angles to the plane of the suture in the tissue annulus. This placement generally prevents plication of the tube between suture platforms 110, while at the same time plicating the tissue annulus itself. In other words, the two needles associated with the mattress suture are turned ninety degrees and placed across, instead of in the same length as, the ring. The needles are brought up at right angles to the long axis of the ring. By placing any additional waist sutures at right angles to the long axis of the tube, such suture may “bunch” the waist or make it tighter, but will not change the overall length of tube 120. Placing waist sutures longitudinally, on the other hand, would tend to shorten the length of tube 120 and the circumference of the resulting ring; the waist would tend to be narrowed slightly in the wrong direction.

Alternatively, the two ends of the suture can simply be tied around the waist in a radial direction, between suture platforms 110. These extra sutures, which are not directly attached or supported by suture platforms 110, allow for additional fixation points between the anatomical structure and system 100, providing additional security. A surgeon also can place an extra suture radially in the tissue annulus or other anatomical structure as a simple suture, if desired.

According to another feature of the invention, one of the platforms 110 is defined as middle suture platform 200. Middle suture platform 200 is enclosed within middle pocket 210 of the tube formed of covering material 120. According to one embodiment, covering material 120 at middle suture pocket 210 is of a contrasting color relative to the remainder of the tube. This color scheme, or an additional or alternative external marker, e.g. a white fixation/guide suture instead of a black one, enables the surgeon to better orient and more accurately deliver tube 120 to the anatomical structure.

According to additional embodiments, covering material 120 is constructed to be readily cut between platforms 110, such that the resulting tube formed by covering material 120 and platforms 110 is of a desired length before being introduced to or secured with respect to the anatomical structure. In the view of FIG. 5, for example, a cut can be made with a scissors, scalpel or other sharp object immediately to the left or right of a desired intersegmental suture 190. One of the free opposite ends of tube 120 thus is separated from the remainder of the tube and can be discarded, recycled or put to other use.

Covering material 120 is constructed and arranged to maintain discrete suture support platforms 110 in a desired overall shape, e.g. a linear or curved shape, but to allow relative movement between adjacent suture support platforms 110 to accommodate the particular shape of the anatomical structure in question. When disposed in a linear shape, the plurality of discrete suture support platforms 110 and covering material 120 are arranged in a line, such that apparatus 100 has opposite ends that are free of direct connection to each other.

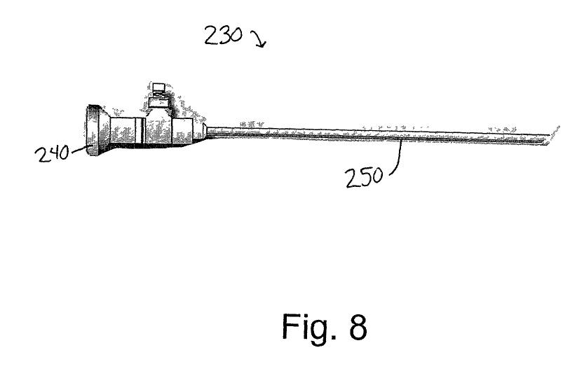

As mentioned previously, embodiments of the invention are especially advantageous for use with a small-diameter endoscope, e.g. in an endoscopic or endoscopic/robotic annuloplasty procedure. As shown in FIG. 8, one such endoscope 230 includes eye piece 240 and rigid or flexible probe 250, connected to a suitable port for insertion of apparatus 100. Apparatus 100 can be introduced through endoscope 200 to the region of the anatomical structure merely by ensuring that apparatus 100 is in a relatively linear configuration. Apparatus 100 need not be compressed, narrowed, disassembled or otherwise altered to fit through endoscope 230. In this way, it is uniquely suited to endoscopic/robotic minimally invasive cardiac surgery, for example. In contrast, many conventional annuloplasty rings are mounted on templates or holders and have resulting dimensions of e.g. about 25 mm by about 30 mm or some other dimension unsuitably large for insertion through endoscope 230 without detaching the ring from the template. Thus, embodiments of the invention provide significant advantages over conventional rings and templates.

Embodiments of the invention also can be used with a deformable/bendable sizer-delivery system like that disclosed in e.g. U.S. Pat. No. 5,961,539, incorporated by reference above. Belts, sutures or other devices can be used to release apparatus 100 from the individual segment holders, such that the two remaining portions of the belt/suture loop are still attached to the holder for removal. A sizer/delivery system according to the above-referenced patent is also especially desirable for endoscopic/robotic practice.

In the case of an annuloplasty procedure, apparatus 100 ultimately can provide a scaffold for e.g. a biomaterial (e.g. untreated autologus paracardiam) to completely cover apparatus 100. Alternatively, other materials can be used, or no material, according to the particular surgical situation involved.

Aspects of the invention provide a number of advantages over other devices and methods for stabilizing and/or reducing the circumference of an anatomical structure, such as certain annuloplasty rings. Aspects of the invention, for example, provide extreme precision in effecting repair of a mitral or tricuspid heart valve, and can be completely fabricated at a manufacturing facility prior to marketing. The surgeon in the operating room does not complete or “build” a ring from bare segments; instead, the segments are spaced and delivered as a unit in the tube. The tube can be cut to a desired length to suit a particular surgical situation.

With the invention, the anterior mitral or tricuspid valve leaflet, for example, can be recognized as a unique, advantageous template for repairing a dilated tissue annulus. Each dilated tissue annulus is brought to a precise anterior mitral or tricuspid leaflet perimeter dimension, rather than to the dimension of a specific-size annuloplasty ring, which itself is an approximation or “best fit” with the anterior leaflet. Surgeons are thus encouraged to begin with the exact perimeter dimension of each unique, unfurled anterior leaflet size and shape particular to a specific patient. Embodiments of the invention allow for an absolutely precise measured plication of the dilated annulus in every case, and allow for absolute precision and accuracy in the measurement of the linear dimension of the annuloplasty ring-equivalent material. The result is valve repair with greater predictability and reproduceability for every unique case, in a manner believed impossible with any of the commonly marketed annuloplasty rings.

The customizability of the invention appeals to the artistry and creativity of the individual surgeon. Annuloplasty rings of appropriate length can be created in all cases, even with an anterior mitral or tricuspid leaflet of unusual size and/or shape. Either a partial or complete annuloplasty ring can be created. Additionally, alternative templates, other than the anterior mitral or tricuspid leaflet, readily can be used.

Aspects of the invention also present significant advantages because of their simplicity in design, manufacture and use. Aspects of the invention allow hospital inventories to stock only one size tube, because one size can be cut or otherwise adjusted to fit all cases. Additionally, hospital inventories do not require separate sizers of various dimensions, since embodiments of the invention also can act as a disposable universal sizer.

Other advantages over commonly available rings and other devices include:

A remodeling annuloplasty can be accomplished without involving the anterior mitral or septal tricuspid annulus, in most cases.

Normal geometry and physiology of the mitral or tricuspid apparatus can be maintained, with 3-dimensional flexibility during the cardiac cycle. Contractility of the mitral or tricuspid apparatus is preserved.

Optimal surface area of the mitral or tricuspid orifice is conserved.

Compatibility with any pathological condition requiring annuloplasty is allowed.

Stresses on sutures are reduced, by maintaining flexibility throughout the cardiac cycle.

An optimal relationship between the ring orifice area and the amount of valvular tissue is allowed, preventing left ventricular outflow tract obstruction.

Fixation of the diastolic dimension of the mitral or tricuspid annulus is allowed, while preserving its normal, flexible contour.

Embodiments of the invention reduce the circumference of an anatomical structure by promoting tissue plication in precise regions. Alternatively, in non-plicating embodiments, the invention can be used merely to stabilize the circumference of such anatomical structures, preventing dilatation or other abnormality. Embodiments of the invention have particular application to vascular structures, such as mitral or tricuspid heart valves, but the invention is by no means limited to these embodiments. A wide variety of other anatomical structures can also be repaired according to embodiments of the invention.

The invention should not be considered limited to the specific methods and devices precisely described herein. On the contrary, various modifications will be apparent to those of ordinary skill upon reading this disclosure. For example, although certain embodiments are described with reference to mitral valves, use with tricuspid or other valves or anatomical structures is also contemplated. Additionally, devices described herein can be made of disposable material, for one-time use, or of non-disposable material, for re-sterilization and subsequent reuse. A single piece or more than one piece of fabric or other material can be used to constitute the disclosed tube. Different colors or other marker/identification schemes can be used to highlight e.g. the middle suture platform, the intersegmental sutures, fixation/guide sutures, etc., and/or to guide suture placement through each pocket/link of the tube and its corresponding suture platform. More than one suture platform can be enclosed within a particular tube link, and each segment can include two or more holes or even a single hole if desired. Any one or more of the sutures shown herein can be of polyester, wire, or other suitable material. Embodiments of the invention have particular application to minimally invasive surgical techniques and/or robotic endoscopic procedures, but also can be used in conventional surgical settings. Other modifications will be apparent to those of ordinary skill.

What is claimed is:

1. Apparatus for stabilizing an anatomical structure, the apparatus comprising:
a plurality of discrete suture support platforms, the plurality of discrete suture support platforms being constructed and arranged for disposition at the anatomical structure to stabilize the anatomical structure; and
covering material constructed and arranged to cover the plurality of discrete suture support platforms, the covering material being formed into a plurality of pockets, at least one discrete suture support platform being contained within each pocket;
wherein each suture support platform is constructed and arranged to receive and support suture extending from the platform through the covering material to secure the platform with respect to the anatomical structure at a precise location.

2. The apparatus of claim 1, wherein the covering material comprises a segmented tube, each segment of the tube corresponding to one of said pockets.

3. The apparatus of claim 1, wherein the covering material is constructed and arranged to maintain the discrete suture support platforms in a desired overall shape but to allow relative movement between adjacent suture support platforms to accommodate the particular shape of the anatomical structure.

4. The apparatus of claim 1, wherein the plurality of discrete suture support platforms and covering material are arranged in a line such that the apparatus has opposite ends that are free of direct connection to each other.

5. The apparatus of claim 4, wherein the discrete suture support platforms each define at least one aperture therethrough, the at least one aperture of each suture support platform being constructed to receive and support suture for attaching each platform with respect to the anatomical structure at a precise location; further wherein the apertures of the platforms are constructed and arranged to create a plication region in the anatomical structure beneath each platform so as to reduce the length of the anatomical structure by a precise amount along the line.

6. The apparatus of claim 4, wherein the covering material is constructed to be cut between platforms such that the line is of a desired length before being secured with respect to the anatomical structure.

7. The apparatus of claim 1, wherein the discrete suture support platforms each define at least one aperture therethrough, the at least one aperture of each suture support platform being constructed to receive and support suture for attaching each platform with respect to the anatomical structure at a precise location, such that the anatomical structure is stabilized.

8. The apparatus of claim 1, wherein said suture is first suture; further wherein the covering material is attached to each discrete suture support platform by second suture.

9. The apparatus of claim 8, wherein said second suture passes through at least one aperture of the suture platform.

10. The apparatus of claim 9, wherein said second suture is tied in a knot, the knot being disposed directly adjacent the at least one aperture to indicate location of the at least one aperture.

11. The apparatus of claim 8, wherein said second suture is disposed and arranged to guide the placement of said first suture through the covering material.

12. The apparatus of claim 8, further comprising third suture constructed and arranged between the suture support platforms to form the pockets; wherein the third suture is of a contrasting color relative to said covering material.

13. The apparatus of claim 1, the apparatus being in combination with said suture.

14. The apparatus of claim 1, wherein said pockets are all generally identical; further wherein said platforms are all generally identical.

15. The apparatus of claim 1, wherein each pocket is a completely closed pocket.

16. The apparatus of claim 1, wherein each suture support platform comprises two apertures therethrough; further wherein the covering material supports a visual indication of the location of the apertures.

17. The apparatus of claim 1, wherein the visual indication comprises suture.

18. Apparatus for stabilizing an anatomical structure, the apparatus being introduced to the anatomical structure during a surgical procedure, the apparatus comprising:
a tube having free opposite ends; and
a plurality of discrete suture platforms disposed within and secured with respect to the tube before the surgical procedure begins, each discrete suture platform being constructed and arranged to support suture.

19. The apparatus of claim 18, wherein an outer surface of the tube defines at least one narrowed portion between each suture platform.

20. The apparatus of claim 18, wherein the tube is constructed to be readily cut to define a desired length of the tube, such that one of the free opposite ends is separated from the remainder of the tube.

21. The apparatus of claim 18, constructed and arranged to be introduced through a small-diameter endoscope.

22. The apparatus of claim 21, wherein the apparatus can be introduced through a small-diameter endoscope without being narrowed, disassembled or otherwise altered.

23. The apparatus of claim 18, further comprising bonding agent to secure each suture platform with respect to the tube.

24. The apparatus of claim 18, wherein the tube defines at least one external guide feature arranged to accurately guide and direct suture placement with respect to each suture platform.

25. The apparatus of claim 24, wherein the at least one external guide feature comprises suture.

26. The apparatus of claim 25, wherein said suture holds each platform in place inside the tube.

27. The apparatus of claim 24, wherein the at least one external guide feature comprises a color contrast.

28. The apparatus of claim 24, wherein the at least one external guide feature indicates location of an aperture within each platform.

29. The apparatus of claim 18, wherein one of the suture platforms is a middle suture platform of the apparatus; further wherein the tube at the middle suture platform is of a contrasting color to the remainder of the tube.

30. The apparatus of claim 18, further comprising sizing features disposed along the tube, wherein the sizing features are constructed and arranged to indicate the length of the anatomical structure based on the number of sizing features needed to extend along an entire desired portion of the anatomical structure.

31. The apparatus of claim 30, wherein the sizing features comprise suture.

32. The apparatus of claim 18, wherein the tube comprises polyester.

33. The apparatus of claim 18, wherein each discrete suture platform is constructed and arranged to receive and support suture passing through the tube and into the anatomical structure such that the tube is secured with respect to and stabilizes the anatomical structure.

34. The apparatus of claim 33, wherein the tube defines a waist between each suture platform; the apparatus being constructed and arranged to receive and support suture passing through at least one waist of the tube and into the anatomical structure such that the tube is additionally secured with respect to and additionally stabilizes the anatomical structure.

35. The apparatus of claim 34, wherein said suture passing through the at least one waist of the tube is arranged to plicate the anatomical structure without plicating the tube.

36. The apparatus of claim 18, constructed and arranged for use in an annuloplasty procedure.

37. In combination:
apparatus for stabilizing an anatomical structure, the apparatus being introduced to the anatomical structure during a surgical procedure, the apparatus comprising:
a tube having free opposite ends; and
a plurality of discrete suture platforms disposed within and secured with respect to the tube before the surgical procedure begins, each discrete suture platform being constructed and arranged to support suture; and
an endoscope for receiving and introducing said apparatus during the surgical procedure.

38. The combination of claim 37, wherein the endoscope is constructed and arranged for use during a robotic endoscopic procedure.
